SEQUENCE LISTING (6 sequences)

SEQ ID NO: 1 - DNA, 1563 bp, Artificial Sequence
  P. raistrickii steroid 15|A-hydroxylase gene PRH
atggctgtcctcaccgaattgttccatagtccttatgtggttcagggggtgtgcgttgccttgctggctgttttcataacaagcatttggggcgatatagtggatgaaatcccccaccgtcgtatacctttagttggcaaaactggctgggagttcaccaacaaaaaggcaaagtcgcgatttactgagtcatgccgcgatctgatcgccgaagggtttgcaaagggagccagcgcttttcaaatcattggagcaacacaaccgatcattgttctgcacccgaagtatatcgatgaaatcaagagccacccagacttgagttttgccgatgccgtcaaaaagatgttcttctctaaccgtgttccgggctttgagccattccacagtggaacggcgatgaatgttaccgtcgaggttgtgcgcaccaagctgacccaagcgcttggatatttgtcaatcccgctttcaaaagaatcatctttgaccttgaaagaagccttgcctcccactgacgattggactccatacaactttgcgcacaagattccgtatatggtggcccgtgtctcatcattagtattcgtaggtgagaccatctgccacaacgatgaatggatcgatgtgtcagtcaactacgcccttgactcctttaaggccatgcgcgagctacgaacatggccatctgtcctccgcccaattgttcattggtttttgccttctactcaaaagctgcgcagtcacttaaagaaagccaacagtctcatcagccacgagattgacagacgagcgttgattcgggaaggcaaactcccagccgaaaacccgccacacaagcctgatgcattggactggtttcgggagactgcggaggcccagagcaacttcactattgaccaggcccgctctcaggtcggactggccttggcagccattcatactacgtccaacttgatcactaatgttatgtatgatctcgccgcatacccagagtatttccagccactacgtgatgagatccaagcggttgctgccgaggatggcgtactcaagaagactagcttgcttaagttcaaactgatggatagtatcatgaaggagtcgcagagaactcatccattatcattgatctccttgaaccgcgtcactcatcagaaggttgttctttccgacggcaccgtgcttcccaaaggcgcaaatgtcgctatttctacaagccctctggaagacgataatgtataccccaacgccgccacctatgatggctaccggttcttgaagaaacgtcaggcaccaggaaacgagcacaaacatcaatttgtcacaacgaccaaggaacacttcgtcttcggccacggtgtccacgcctgcccgggtcgtttcttcgctgccaatgaaaccaaaatcctactccttcatctgctgttgaagtacgactggaaattgcagagtggtggacgaccgaaaaattttagcaatggcaccgagtctattaccgaccccacggttgaactactgttccggtctcgtgaacccgagattgacttgagtttcttgggagagtag

SEQ ID NO: 2 - DNA, 1518 bp, Artificial Sequence
  S. cerevisiae G6PD gene ZWF1
atgagtgaaggccccgtcaaattcgaaaaaaataccgtcatatctgtctttggtgcgtcaggtgatctggcaaagaagaagacttttcccgccttatttgggcttttcagagaaggttaccttgatccatctaccaagatcttcggttatgcccggtccaaattgtccatggaggaggacctgaagtcccgtgtcctaccccacttgaaaaaacctcacggtgaagccgatgactctaaggtcgaacagttcttcaagatggtcagctacatttcgggaaattacgacacagatgaaggcttcgacgaattaagaacgcagatcgagaaattcgagaaaagtgccaacgtcgatgtcccacaccgtctcttctatctggccttgccgccaagcgtttttttgacggtggccaagcagatcaagagtcgtgtgtacgcagagaatggcatcacccgtgtaatcgtagagaaacctttcggccacgacctggcctctgccagggagctgcaaaaaaacctggggcccctctttaaagaagaagagttgtacagaattgaccattacttgggtaaagagttggtcaagaatcttttagtcttgaggttcggtaaccagtttttgaatgcctcgtggaatagagacaacattcaaagcgttcagatttcgtttaaagagaggttcggcaccgaaggccgtggcggctatttcgactctataggcataatcagagacgtgatgcagaaccatctgttacaaatcatgactctcttgactatggaaagaccggtgtcttttgacccggaatctattcgtgacgaaaaggttaaggttctaaaggccgtggcccccatcgacacggacgacgtcctcttgggccagtacggtaaatctgaggacgggtctaagcccgcctacgtggatgatgacactgtagacaaggactctaaatgtgtcacttttgcagcaatgactttcaacatcgaaaacgagcgttgggagggcgtccccatcatgatgcgtgccggtaaggctttgaatgagtccaaggtggagatcagactgcagtacaaagcggtcgcatcgggtgtcttcaaagacattccaaataacgaactggtcatcagagtgcagcccgatgccgctgtgtacctaaagtttaatgctaagacccctggtctgtcaaatgctacccaagtcacagatctgaatctaacttacgcaagcaggtaccaagacttttggattccagaggcttacgaggtgttgataagagacgccctactgggtgaccattccaactttgtcagagatgacgaattggatatcagttggggcatattcaccccattactgaagcacatagagcgtccggacggtccaacaccggaaatttacccctacggatcaagaggtccaaagggattgaaggaatatatgcaaaaacacaagtatgttatgcccgaaaagcacccttacgcttggcccgtgactaagccagaagatacgaaggataattag

SEQ ID NO: 3 - DNA, 28 bp, Artificial Sequence
  PrimerPRH-F
ggaattcatggctgtcctcaccgaattg

SEQ ID NO: 4 - DNA, 33 bp, Artificial Sequence
  PrimerPRH-R
ataagaatgcggccgcctactctcccaagaaac

SEQ ID NO: 5 - DNA, 31 bp, Artificial Sequence
  PrimerZWF1-F
ggaattcagtagtgaaggccccgtcaaattc

SEQ ID NO: 6 - DNA, 33 bp, Artificial Sequence
  PrimerZWF1-R
gctctagactaattatccttcgtatcttgtggc

(19) United States
(12) Patent Application Publication
(10) Pub. No.: US 2021/0355457 A1
(43) Pub. Date: Nov. 18, 2021

(54) RECOMBINANT PICHIA PASTORIS, CONSTRUCTION METHOD THEREOF, AND USE THEREOF IN EFFICIENT PREPARATION OF 15 ALPHA-D-ETHYLGONENDIONE

(71) Applicant: Tianjin University of Science and Technology, Tianjin (CN)

(72) Inventors: Xiaoguang Liu, Tianjin (CN); Fuping Lu, Tianjin (CN); Shuhong Mao, Tianjin (CN); Peng Jin, Tianjin (CN); Dongqi Xie, Tianjin (CN); Yanxi Chen, Tianjin (CN); Jinhong Li, Tianjin (CN)

(73) Assignee: Tianjin University of Science and Technology, Tianjin (CN)

(21) Appl. No.: 17/318,934

(22) Filed: May 12, 2021

(30) Foreign Application Priority Data
May 13, 2020 (CN) 202010402827.0

(51) Int. Cl. C12N 9/02; C12N 9/04; C12P 33/00

(52) U.S. Cl. CPC C12N 9/0071; C12P 33/00; C12N 9/0006; C12Y 101/01049

(57) ABSTRACT

The present disclosure provides recombinant Pichia pastoris, a construction method thereof, and use thereof in the efficient preparation of 15α-D-ethylgonendione, and belongs to the technical field of fermentation engineering. In the present disclosure, with Pichia pastoris as a host, 15α-steroid hydroxylase and glucose-6-phosphate dehydrogenase (G6PD) are simultaneously induced in a manner of intracellular enzyme production for the first time, and the Pichia pastoris whole-cell induction and catalysis mode is used to achieve the conversion of D-ethylgonendione with high substrate loading and high conversion rate.

### CROSS REFERENCE TO RELATED APPLICATION

This patent application claims the benefit and priority of Chinese Patent Application No. 202010402827.0, filed on May 13, 2020, the disclosure of which is incorporated by reference herein in its entirety as part of the present application

### TECHNICAL FIELD

The present disclosure relates to the technical field of fermentation engineering, and in particular to recombinant Pichia pastoris, a construction method thereof, and use thereof in the efficient preparation of 15α-D-ethylgonendione.

### BACKGROUND ART

Steroidal drugs are an important class of drugs with a wide range of pharmacological and physiological activities, which play an extremely important role in anti-inflammation, anti-allergy, anti-tumor, birth control, and the like. As one of the main components for third-generation powerful contraceptives, gestodene is the most ideal oral contraceptive by far.

The chemical synthesis of gestodene requires the use of expensive catalysts Pb(OAc)₂ and Bu₃SnOMe, which results in high cost and heavy pollution. Microbial biotransformation is widely used in the steroidal compound industry due to its high regioselectivity, stereoselectivity, and environmental friendliness. 15α-D-ethylgonendione is a key intermediate for the synthesis of gestodene. At present, hydroxyl is introduced at a C15 site of D-ethylgonendione by the specific enzymatic reaction of a Penicilliun raistrickii (P. raistrickii) steroid 15α-hydroxylase to obtain 15α-D-ethylgonendione industrially.

In actual industrial production, the P. raistrickii-mediated 15α-hydroxylation of D-ethylgonendione faces issues such as low substrate loading (2 g/L), long conversion time (72 h), and so on, which severely restricts a conversion rate of 15α-D-ethylgonendione.

### SUMMARY

The present disclosure is intended to provide recombinant Pichia pastoris and a method for efficiently converting D-ethylgonendione with the recombinant Pichia pastoris. The present disclosure solves the technical problem of low D-ethylgonendione loading and realizes the efficient production of 15α-D-ethylgonendione (an intermediate for gestodene).

To achieve the objective of the present disclosure, the present disclosure provides the following technical solutions.

The present disclosure provides recombinant Pichia pastoris. The recombinant Pichia pastoris is obtained by inserting a P. raistrickii steroid 15α-hydroxylase gene PRH and a Saccharomyces cerevisiae (S. cerevisiae) glucose-6-phosphate dehydrogenase (G6PD) gene ZWF1 into the genome of Pichia pastoris.

The P. raistrickii steroid 15α-hydroxylase gene PRH is inserted into a His4 site of the genome of Pichia pastoris;

the S. cerevisiae G6PD gene ZWF1 is inserted into a 5′AOX1 site of the genome of Pichia pastoris:

the P. raistrickii steroid 15α-hydroxylase gene PRH has a nucleotide sequence shown in SEQ ID NO. 1; and

the S. cerevisiae G6PD gene ZWF1 has a nucleotide sequence shown in SEQ ID NO. 2.

The present disclosure also provides a construction method of the recombinant Pichia pastoris according to the above solution, including the following steps:

S1. inserting the P. raistrickii steroid 15α-hydroxylase gene PRH into pPIC3.5K to obtain a first recombinant plasmid:

S2. inserting the S. cerevisiae G6PD gene ZWF1 into pPICZ to obtain a second recombinant plasmid; and

S3. linearizing the first recombinant plasmid with a restriction endonuclease Sal I and the second recombinant plasmid with a restriction endonuclease Sac I, and electrotransforming the plasmids into Pichia pastoris to obtain the recombinant Pichia pastoris;

where steps S1 and S2 can be conducted in any order.

Preferably, the P. raistrickii steroid 15α-hydroxylase gene PRH may be inserted into the pPIC3.5K at sites of FcoR I and Not I; and the S. cerevisiae G6PD gene ZWF1 may be inserted into the pPICZ at sites of EcoR I and Xba I.

Preferably, the Pichia pastoris in step S3 may include Pichia pastoris GS115.

The present disclosure also provides use of the recombinant Pichia pastoris according to the above solution in the efficient preparation of 15α-D-ethylgonendione, including the following steps:

1) inoculating the recombinant Pichia pastoris into a seed culture medium, and cultivating until a resulting bacterial solution has OD₆₀₀ of 11 to 13 to obtain a starter culture:

2) inoculating the starter culture into a fermentation medium, and conducting fermentation cultivation at a temperature of 28° C. to 30° C. and a dissolved oxygen content of more than 20% until glycerin in the fermentation system is exhausted;

3) after the glycerin in the fermentation system is exhausted, feeding a glycerin-trace element aqueous solution into the fermentation system at a rate of 19.92 mL/(L·h), and stopping the feeding when a dissolved oxygen content is lower than 20%; further conducting fermentation cultivation until glycerin is exhausted once again; and with the glycerin being exhausted, starving bacteria for 1 h, where the glycerin-trace element aqueous solution includes glycerin with a volume percentage of 45% to 55% and a 12 mL/L trace element aqueous solution;

4) after the bacteria are starved for 1 h, feeding a trace element-methanol solution into the fermentation system with a dissolved oxygen content of 100% for the first time at a rate of 3.61 mL/(L·h), and stopping the feeding when a dissolved oxygen content is lower than 20%; conducting a first induction cultivation for 2 h at 28° C. to 30° C.; and feeding the trace element-methanol solution into the fermentation system for the second time at a rate of 7.22 mL/(L·h) for 2 h; and

5) after the trace element-methanol solution is fed for the second time for 2 h, feeding a D-ethylgonendione-methanol solution at a rate of 10.89 mL/(L·h) until the D-ethylgonendione has a concentration of 8 g/L in the fermentation system, and with a dissolved oxygen content being controlled always at above 20%, conducting reaction at 28° C. to 30° C. until the D-ethylgonendione is completely converted:

where the fermentation medium in step 2) includes the following components, with water as a solvent: 85% phosphoric acid: 26.7 mL/L, CaSO₄: 0.93 g/L, K₂SO₄: 18.2 g/L, MgSO₄: 7.27 g/L, KOH: 4.13 g/L, Tween 80: 0.6 mL/L, glycerin: 40 g/L, and a trace element aqueous solution with a volume percentage of 0.43%; and the fermentation medium has a pH of 5.0;

the trace element-methanol solution in step 4) is based on methanol and further includes a 12 mL/L trace element aqueous solution;

the D-ethylgonendione-methanol solution in step 5) is based on methanol and further includes a 12 mL/L trace element aqueous solution and 4 g/L to 5 g/L D-ethylgonendione; and

the trace element aqueous solution includes the following components, with water as a solvent: CuSO₄.5H₂O: 6.0 g/L, NaI: 0.08 g/L, MnSO₄.H₂O: 3.0 g/L, NaMoO₄.2H₂O: 0.2 g/L, H₃BO₃: 0.02 g/L, CoCl₂: 0.5 g/L, ZnCl₂: 20.0 g/L, FeSO₄.7H₂O: 65.0 g/L, biotin: 0.2 g/L, and sulfuric acid: 5.0 mL/L.

Preferably, the seed culture medium in step 1) may be YPG liquid medium.

Preferably, an initial inoculation amount of the starter culture in step 2) may be based on OD₆₀₀ after inoculation; and the OD₆₀₀ may be 0.8 to 1.0.

Beneficial effects of the present disclosure: The present disclosure provides recombinant Pichia pastoris. The recombinant Pichia pastoris is obtained by inserting a P. raistrickii steroid 15α-hydroxylase gene PRH and an S. cerevisiae G6PD gene ZWF7 into a genome of Pichia pastoris. The recombinant Pichia pastoris of the present disclosure can achieve the simultaneous induction of 15α-steroid hydroxylase and G6PD in Pichia pastoris (as a host) in a manner of intracellular enzyme production. The recombinant Pichia pastoris of the present disclosure can achieve the efficient conversion of D-ethylgonendione to efficiently prepare 15α-D-ethylgonendione.

The present disclosure also provides use of the recombinant Pichia pastoris according to the above solution in the efficient preparation of 15α-D-ethylgonendione, including the following steps: recombinant Pichia pastoris seed cultivation, bacterial growth, feed-batch cultivation of bacteria, induced enzyme production, and D-ethylgonendione conversion. The present disclosure uses the Pichia pastoris whole-cell induction and catalysis mode to achieve the conversion of D-ethylgonendione at a high substrate loading as high as 8 g/L, with a conversion rate of 98%. The preparation method has high preparation efficiency, which is 3 times higher than that of the industrial D-ethylgonendione conversion by P. raistrickii.

### BRIEF DESCRIPTION OF DRAWINGS

FIG. 1 is a schematic structural diagram of the recombinant plasmid pPIC3.5K-PRH;

FIG. 2 is a schematic structural diagram of the recombinant plasmid pPICZ-ZWF7;

FIG. 3 is a schematic diagram illustrating the genome of Pichia pastoris GS115 transformed with the recombinant plasmids pPIC3.5K-PRH and pPICZ-ZWF7;

FIG. 4 shows a PCR identification result of the genome of Pichia pastoris GS115 transformed with the recombinant plasmids pPIC3.5K-PRH and pPICZ-ZWF1, where M: 1 kb DNA ladder: 1: ZWF1 gene verification; and 2: PRH gene verification:

FIG. 5 is a thin Layer chromatography (TLC) chart illustrating the conversion of D-ethylgonendione by the recombinant Pichia pastoris; and

FIG. 6 is a high performance liquid chromatography (HPLC) chart illustrating the conversion of D-ethylgonendione by the recombinant Pichia pastoris, where a: D-ethylgonendione; and b: 15α-D-ethylgonendione.

### DETAILED DESCRIPTION

The present disclosure provides recombinant Pichia pastoris. The recombinant Pichia pastoris is obtained by inserting a P. raistrickii steroid 15α-hydroxylase gene PRH and an S. cerevisiae G6PD gene ZWF1 into a genome of Pichia pastoris. The P. raistrickii steroid 15α-hydroxylase gene PRH is inserted into a His4 site of the genome of Pichia pastoris: the S. cerevisiae G6PD gene ZWF1 is inserted into a 5′AOX1 site of the genome of Pichia pastoris; the P. raistrickii steroid 15α-hydroxylase gene PRH has a nucleotide sequence shown in SEQ ID NO. 1; and the S. cerevisiae G6PD gene ZWF1 has a nucleotide sequence shown in SEQ ID NO. 2.

The P. raistrickii steroid 15 α-hydroxylase gene PRH has a nucleotide sequence shown in SEQ ID NO. 1, specifically as follows:

ATGGCTGTCCTCACCGAATTGTTCCATAGTCCTTATGTGGTTCAGGGGGT
GTGCGTTGCCTTGCTGGCTGTTTTCATAACAAGCATTTGGGGCGATATAG
TGGATGAAATCCCCCACCGTCGTATACCTTTAGTTGGCAAAACTGGCTGG
GAGTTCACCAACAAAAAGGCAAAGTCGCGATTTACTGAGTCATGCCGCGA
TCTGATCGCCGAAGGGTTTGCAAAGGGAGCCAGCGCTTTTCAAATCATTG
GAGCAACACAACCGATCATTGTTCTGCACCCGAAGTATATCGATGAAATC
AAGAGCCACCCAGACTTGAGTTTTGCCGATGCCGTCAAAAAGATGTTCTT
CTCTAACCGTGTTCCGGGCTTTGAGCCATTCCACAGTGGAACGGCGATGA
ATGTTACCGTCGAGGTTGTGCGCACCAAGCTGACCCAAGCGCTTGGATAT
TTGTCAATCCCGCTTTCAAAAGAATCATCTTTGACCTTGAAAGAAGCCTT
GCCTCCCACTGACGATTGGACTCCATACAACTTTGCGCACAAGATTCCGT
ATATGGTGGCCCGTGTCTCATCATTAGTATTCGTAGGTGAGACCATCTGC
CACAACGATGAATGGATCGATGTGTCAGTCAACTACGCCCTTGACTCCTT
TAAGGCCATGCGCGAGCTACGAACATGGCCATCTGTCCTCCGCCCAATTG
TTCATTGGTTTTTGCCTTCTACTCAAAAGCTGCGCAGTCACTTAAAGAAA
GCCAACAGTCTCATCAGCCACGAGATTGACAGACGAGCGTTGATTCGGGA
AGGCAAACTCCCAGCCGAAAACCCGCCACACAAGCCTGATGCATTGGACT
GGTTTCGGGAGACTGCGGAGGCCCAGAGCAACTTCACTATTGACCAGGCC
CGCTCTCAGGTCGGACTGGCCTTGGCAGCCATTCATACTACGTCCAACTT
GATCACTAATGTTATGTATGATCTCGCCGCATACCCAGAGTATTTCCAGC
CACTACGTGATGAGATCCAAGCGGTTGCTGCCGAGGATGGCGTACTCAAG
AAGACTAGCTTGCTTAAGTTCAAACTGATGGATAGTATCATGAAGGAGTC
GCAGAGAACTCATCCATTATCATTGATCTCCTTGAACCGCGTCACTCATC
AGAAGGTTGTTCTTTCCGACGGCACCGTGCTTCCCAAAGGCGCAAATGTC
GCTATTTCTACAAGCCCTCTGGAAGACGATAATGTATACCCCAACGCCGC
CACCTATGATGGCTACCGGTTCTTGAAGAAACGTCAGGCACCAGGAAACG
AGCACAAACATCAATTTGTCACAACGACCAAGGAACACTTCGTCTTCGGC
CACGGTGTCCACGCCTGCCCGGGTCGTTTCTTCGCTGCCAATGAAACCAA
AATCCTACTCCTTCATCTGCTGTTGAAGTACGACTGGAAATTGCAGAGTG
GTGGACGACCGAAAAATTTTAGCAATGGCACCGAGTCTATTACCGACCCC
ACGGTTGAACTACTGTTCCGGTCTCGTGAACCCGAGATTGACTTGAGTTT
CTTGGGAGAGTAG.

In the present disclosure, the recombinant Pichia pastoris provides the 15α hydroxylation function of P. raistrickii and catalyzes the formation of 15α-D-ethylgonendione from D-ethylgonendione. The S. cerevisiae G6PD gene ZWF1 has a nucleotide sequence shown in SEQ ID NO. 2, specifically as follows:

ATGAGTGAAGGCCCCGTCAAATTCGAAAAAAATACCGTCATATCTGTCTITGGTG CGTC AGGTGATCTGGCAAAGAAGAAGACTITTCCCGCCTTATTGGGCTTTTCAGAGAA GGTTACCTTGATCCATCTACCAAGATCTTCGGTTATGCCCGGTCCAAATTGTCCATGGAG GAGGACCTGAAGTCCCGTGTCCTACCCCACTTGAAAAAACCTCACGGTGAAGCCGATG ACTCTAAGGTCGAACAGTTCTTCAAGATGGTCAGCTACATTTCGGGAAATTACGACACA GATGAAGGCTTCGACGAATTAAGAACGCAGATCGAGAAATTCGAGAAAAGTGCCAACG TCGATGTCCCACACCGTCTCTTCTATCTGGCCTTGCCGCCAAGCGTTTTTTTGACGGTGG CCAAGCAGATCAAGAGTCGTGTGTACGCAGAGAATGGCATCACCCGTGTAATCGTAGAG AAACCTTTCGGCCACGACCTGGCCTCTGCCAGGGAGCTGCAAAAAAACCTGGGGCCCC TCTTTAAAGAAGAAGAGTTGTACAGAATTGACCATTACTTGGGTAAAGAGTTGGTCAAG AATCTTTTAGTCTTGAGGTTCGGTAACCAGTTTTTGAATGCCTCGTGGAATAGAGACAAC ATTCAAAGCGTTCAGATTTCGTTTAAAGAGAGGTTCGGCACCGAAGGCCGTGGCGGCTA TTTCGACTCTATAGGCATAATCAGAGACGTGATGCAGAACCATCTGTTACAAATCATGAC TCTCTTGACTATGGAAAGACCGGTGTCTITTGACCCGGAATCTATTCGTGACGAAAAGG TTAAGGTTCTAAAGGCCGTGGCCCCCATCGACACGGACGACGTCCTCTTGGGCCAGTAC GGTAAATCTGAGGACGGGTCTAAGCCCGCCTACGTGGATGATGACACTGTAGACAAGGA CTCTAAATGTGTCACTTTTGCAGCAATGACTfTCAACATCGAAAACGAGCGTTGGGAGG GCGTCCCCATCATGATGCGTGCCGGTAAGGCTTTGAATGAGTCCAAGGTGGAGATCAGA CTGCAGTACAAAGCGGTCGCATCGGGTGTCTTCAAAGACATTCCAAATAACGAACTGGT CATCAGAGTGCAGCCCGATGCCGCTGTGTACCTAAAGTITAATGCTAAGACCCCTGGTCT GTCAAATGCTACCCAAGTCACAGATCTGAATCTAACTTACGCAAGCAGGTACCAAGACT TTTGGATTCCAGAGGCTTACGAGGTGTTGATAAGAGACGCCCTACTGGGTGACCATTCC AACTTTGTCAGAGATGACGAATTGGATATCAGTTGGGGC ATATTCACCCCATTACTGAAG CACATAGAGCGTCCGGACGGTCCAACACCGGAAATITACCCCTACGGATCAAGAGGTCC AAAGGGATTGAAGGAATATATGCAAAAACACAAGTATGTTATGCCCGAAAAGCACCCTT ACGCTTGGCCCGTGACTAAGCCAGAAGATACGAAGGATAATTAG. The present disclosure uses the G6PD gene ZWF1 to reduce NADP⁺ in the recombinant Pichia pastoris into NADPH, thus providing sufficient NADPH for the recombinant Pichia pastoris to catalyze the conversion of D-ethylgonendione.

In the present disclosure, the P. raistrickii steroid 15α-hydroxylase gene PRH and the S. cerevisiae G6PD gene ZWF1 are obtained by a conventional PCR amplification method in the art, and the recombinant Pichia pastoris refers to a positive strain screened out by a conventional Pichia pastoris screening method in the art.

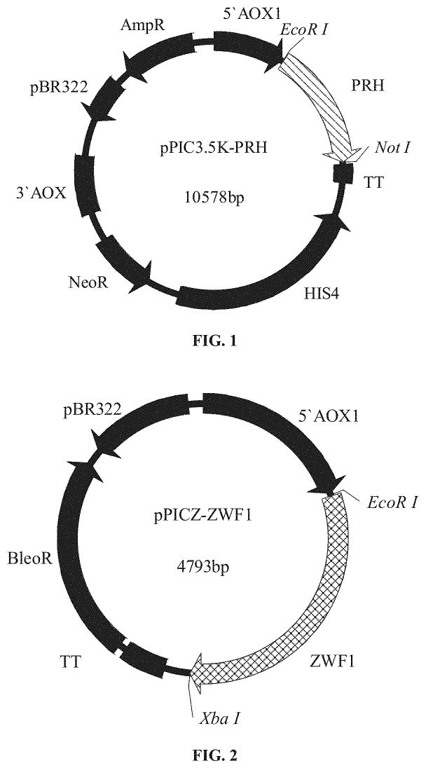

In the present disclosure, a schematic structural diagram of the recombinant plasmid pPIC3.5K-PRH is shown in FIG. 1; and a schematic structural diagram of the recombinant plasmid pPICZ-ZWF1 is shown in FIG. 2.

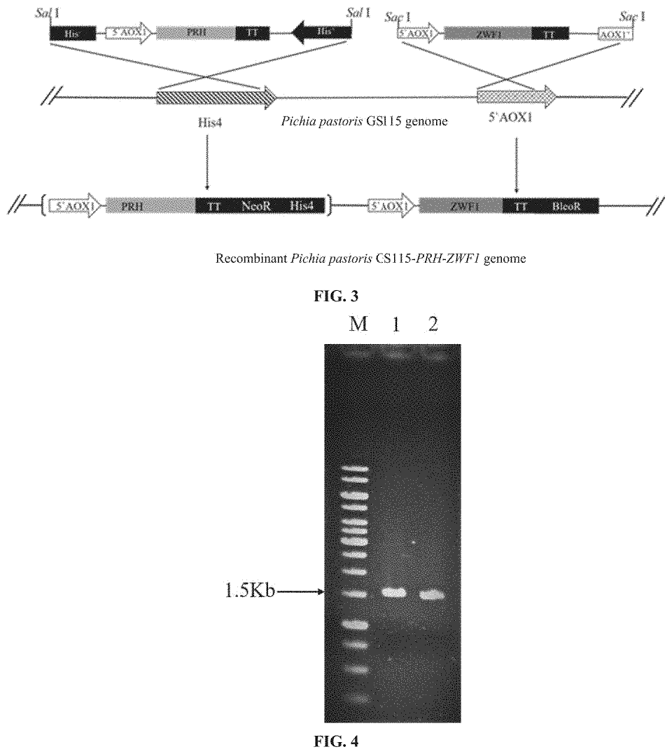

The present disclosure also provides a construction method of the recombinant Pichia pastoris according to the above solution as shown in FIG. 3, including the following steps:

S1. inserting the P. raistrickii steroid 15α-hydroxylase gene PRH into pPIC3.5K to obtain a first recombinant plasmid;

S2. inserting the S. cerevisiae G6PD gene ZWF1 into pPICZ to obtain a second recombinant plasmid; and

S3. linearizing the first recombinant plasmid with a restriction endonuclease Sal I and the second recombinant plasmid with a restriction endonuclease Sac I, and electrotransforming the plasmids into Pichia pastoris to obtain the recombinant Pichia pastoris;

where steps S1 and S2 can be conducted in any order.

In the present disclosure, the P. raistrickii steroid 15α-hydroxylase gene PRH is inserted into pPIC3.5K to obtain a first recombinant plasmid. The vector pPIC3.5K for intracellular expression in Pichia pastoris is preferred for the exogenous expression of the P. raistrickii gene PRH. The geneticin resistance carried on the pPIC3.5K is conducive to the screening of high-copy PRH recombinant strains. The P. raistrickii steroid 15α-hydroxylase gene PRH may be inserted into the pPIC3.5K preferably at sites of EcoR I and Not I. Specifically:

The PRH gene obtained from amplification and pPIC3.5K are digested with EcoR I and Not I enzymes, separately, and ligation is conducted with T4 ligase. A ligation system (10 μL in total) includes: 2 μL of target fragment; 1 μL of pPIC3.5K fragment, 1 μL 10×T4 DNA Ligase buffer; 1 μL of T4 DNA Ligase; and the balance of ddH₂O. The above system is subjected to ligation in a 22° C. water bath for 1 h to 2 h, and a product is transformed into competent Escherichia coli (E. coli) JM109. The plasmids are extracted and verified by restriction enzyme digestion. Restriction enzyme digestion systems are as follows:

a) Single-Enzyme Digestion Verification

A reaction system is 10 μL in total and includes the following components: 2 μL of recombinant plasmid; 1 μL of 10×Buffer; 0.5 μL of enzyme EcoR I; and 6.5 μL of ddH₂O. Reaction is conducted at 37° C. for 2 h. and then 0.8% agarose gel electrophoresis is conducted.

b) Double-Enzyme Digestion Verification

A reaction system is 10 μL in total and includes the following components: 2 μL of recombinant plasmid: 1 μL of 10×Buffer; 0.5 μL of enzyme EcoR I; 0.5 μL of enzyme Not I; and 6 μL of ddH₂O. Reaction is conducted at 37° C. for 2 h, and then 0.8% agarose gel electrophoresis is conducted.

In the present disclosure, the S. cerevisiae G6PD gene ZWF1 is inserted into pPICZ to obtain a second recombinant plasmid. The vector pPICZ for intracellular expression in Pichia pastoris is preferred for the exogenous expression of the S. cerevisiae G6PD gene ZWF1. The bleomycin resistance carried on the pPICZ is conducive to the screening of positive strains. The S. cerevisiae G6PD gene ZWF1 may be inserted into the pPICZ preferably at sites of EcoR I and Xba I. Specifically:

The ZWF1 gene obtained from amplification and pPICZ are digested with EcoR I and Xba I enzymes, separately, and ligation is conducted with T4 ligase. A ligation system (10 μL in total) includes: 2 μL of target fragment ZWF1; 1 μL of pPICZ fragment, 1 μL 10×T4 DNA Ligase buffer; 1 μL of T4 DNA Ligase; and the balance of ddH₂O. The above system is subjected to ligation in a 22° C. water bath for 1 h to 2 h, and a product is transformed into competent E. coli JM109. The plasmids are extracted and verified by restriction enzyme digestion. Restriction enzyme digestion systems are as follows:

a) Single-Enzyme Digestion Verification

A reaction system is 10 μL in total and includes the following components: 2 μL of recombinant plasmid; 1 μL of 10×Buffer: 0.5 μL of enzyme EcoR I; and 6.5 μL of ddH₂O. Reaction is conducted at 37° C. for 2 h, and then 0.8% agarose gel electrophoresis is conducted.

b) Double-Enzyme Digestion Verification

A reaction system is 10 μL in total and includes the following components: 2 μL of recombinant plasmid; 1 μL of 10×Buffer: 0.5 μL of enzyme EcoR I; 0.5 μL of enzyme Xba I; and 6 μL of ddH₂O. Reaction is conducted at 37° C. for 2 h. and then 0.8% agarose gel electrophoresis is conducted.

In the present disclosure, after the first and second recombinant plasmids are obtained, the first recombinant plasmid is linearized with a restriction endonuclease Sal I and the second recombinant plasmid is linearized with a restriction endonuclease Sac I, and then the plasmids are electrotransformed into Pichia pastoris to obtain the recombinant Pichia pastoris. The Pichia pastoris may include Pichia pastoris GS115.

The present disclosure also provides use of the recombinant Pichia pastoris according to the above solution in the efficient preparation of 15α-D-ethylgonendione, including the following steps:

1) inoculating the recombinant Pichia pastoris into a seed culture medium, and cultivating until a resulting bacterial solution has OD₆₀₀ of 11 to 13 to obtain a starter culture:

2) inoculating the starter culture into a fermentation medium, and conducting fermentation cultivation at a temperature of 28° C. to 30° C. and a dissolved oxygen content of more than 20% until glycerin in the fermentation system is exhausted;

3) after the glycerin in the fermentation system is exhausted, feeding a glycerin-trace element aqueous solution into the fermentation system at a rate of 19.92 mL/(L·h), and stopping the feeding when a dissolved oxygen content is lower than 20%; further conducting fermentation cultivation until glycerin is exhausted once again; and with the glycerin being exhausted, starving bacteria for 1 h, where the glycerin-trace element aqueous solution includes glycerin with a volume percentage of 45% to 55% and a 12 mL/L trace element aqueous solution;

4) after the bacteria are starved for 1 h, feeding a trace element-methanol solution into the fermentation system with a dissolved oxygen content of 100% for the first time at a rate of 3.61 mL/(L·h), and stopping the feeding when a dissolved oxygen content is lower than 20%; conducting a first induction cultivation for 2 h at 28° C. to 30° C.; and feeding the trace element-methanol solution into the fermentation system for the second time at a rate of 7.22 mL/(L·h) for 2 h; and

5) after the trace element-methanol solution is fed for the second time for 2 h, feeding a D-ethylgonendione-methanol solution at a rate of 10.89 mL/(L·h) until the D-ethylgonendione has a concentration of 8 g/L in the fermentation system, and with a dissolved oxygen content being controlled always at above 20%, conducting reaction at 28° C. to 30° C. until the D-ethylgonendione is completely converted:

The fermentation medium in step 2) includes the following components, with water as a solvent: 85% phosphoric acid: 26.7 mL/L, CaSO₄: 0.93 g/L, K₂SO₄: 18.2 g/L, MgSO₄: 7.27 g/L, KOH: 4.13 g/L, Tween 80: 0.6 mL/L, glycerin: 40 g/L, and a trace element aqueous solution with a volume percentage of 0.43%; and the fermentation medium has a pH of 5.0. The Tween 80 can activate cell surface, reduce surface tension, and release energy of the reaction solution, which solubilizes the cell membrane by destroying a phospholipid bilayer and forming mixed particles with membrane components, thereby accelerating the entry of steroid molecules into the cell interior from the cell surface.

The trace element-methanol solution in step 4) is based on methanol and further includes a 12 mL/L trace element aqueous solution.

The D-ethylgonendione-methanol solution in step 5) is based on methanol and further includes a 12 mL/L trace element aqueous solution and 4 g/L to 5 g/L D-ethylgonendione. D-ethylgonendione is hardly soluble in water and thus is dissolved in methanol to improve the solubility.

The trace element aqueous solution includes the following components, with water as a solvent: CuSO₄.5H₂O: 6.0 g/L, NaI: 0.08 g/L, MnSO₄.H₂O: 3.0 g/L, NaMoO₄.2H₂O: 0.2 g/L, H₃BO₃: 0.02 g/L, CoCl₂: 0.5 g/L, ZnCl₂: 20.0 g/L, FeSO₄.7H₂O: 65.0 g/L, biotin: 0.2 g/L, and sulfuric acid: 5.0 mL/L.

In the present disclosure, the recombinant Pichia pastoris is first inoculated into a seed culture medium and cultivated at 30° C. and 200 r/min to 220 r/min until a resulting bacterial solution has OD₆₀₀ of 11 to 13 and preferably of 12 to obtain a starter culture. The seed culture medium may preferably be YPG liquid medium. The YPG liquid medium may include the following components by mass: 20 g of peptone, 10 g of yeast powder, and 20 g of glycerin, and a mixture of the above components are diluted to 1 L and sterilized at 115° for 20 min.

In the present disclosure, after the starter culture is obtained, the starter culture is inoculated into a fermentation medium, and fermentation cultivation is conducted at a temperature of 28° C. to 30° C. and a dissolved oxygen content of more than 20% until glycerin in the fermentation system is exhausted. An initial inoculation amount of the starter culture may be based on OD₆₀₀ after inoculation; and the OD₆₀₀ may be preferably 0.8 to 1.0 and more preferably 0.9. The fermentation cultivation may be conducted preferably at 30° C. In the present disclosure, the dissolved oxygen content of more than 20% is set to ensure sufficient oxygen for maintaining the growth of recombinant Pichia pastoris and the conversion of D-ethylgonendione.

In the present disclosure, after the glycerin in the fermentation system is exhausted, a glycerin-trace element aqueous solution is fed into the fermentation system at a rate of 19.92 mL/(L·h) (this rate allows the added glycerin to be completely consumed within 4 h), and the feeding is stopped when a dissolved oxygen content is lower than 20%; fermentation cultivation is further conducted until glycerin is exhausted once again; and with the glycerin being exhausted, bacteria are starved for 1 h (the complete exhaustion of glycerin is beneficial to methanol induction). The glycerin-trace element aqueous solution includes glycerin with a volume percentage of 45% to 55% and a 12 mL/L trace element aqueous solution. The glycerin may have a volume percentage preferably of 50% in the glycerin-trace element aqueous solution.

In the present disclosure, after the bacteria are starved for 1 h, a trace element-methanol solution is fed into the fermentation system for the first time at a rate of 3.61 mL/(L·h), and the feeding is stopped wjen a dissolved oxygen content is lower than 20%; a first induction cultivation is conducted for 2 h at 28° C. to 30° C.; and the trace element-methanol solution is fed into the fermentation system for the second time at a rate of 7.22 mL/(L·h) for 2 h.

In the present disclosure, after the trace element-methanol solution is fed for the second time for 2 h, a D-ethylgonendione-methanol solution is fed at a rate of 10.89 mL/(L·h) until the D-ethylgonendione has a concentration of 8 g/L in the fermentation system; and reaction is conducted at 28° C. to 30° C. until the D-ethylgonendione is completely converted, during which period, a sample is collected and tested every 8 h.

In the present disclosure, methanol is adopted as a solvent of D-ethylgonendione. Methanol can induce the recombinant Pichia pastoris to produce 15a hydroxylase and G6PD and can also improve the solubility of D-ethylgonendione. D-ethylgonendione enters into the fermentation system with the feeding of methanol, such that conversion and induction can be achieved at the same time, which can significantly improve the conversion efficiency.

In the present disclosure, the D-ethylgonendione-methanol solution is fed at a rate of 10.89 mL/(L·h) to enable bacterium growth, methanol-induced enzyme production, and D-ethylgonendione conversion at the optimal state.

In the present disclosure, TLC or HPLC is adopted to detect whether the D-ethylgonendione is completely converted.

The technical solutions of the present disclosure will be clearly and completely described below with reference to the examples of the present disclosure. Apparently, the described examples are merely some rather than all of the examples of the present disclosure. All other examples obtained by a person of ordinary skill in the art based on the examples of the present disclosure without creative efforts shall fall within the protection scope of the present disclosure.

The examples involved the following primer sequences:

PRH-F:
GGAATTCATGGCTGTCCTCACCGAATTG,
as shown in SEQ ID NO. 3;
PRH-R:
ATAAGAATGCGGCCGCCTACTCTCCCAAGAAAC,
as shown in SEQ ID NO. 4;
ZWF1-F:
GGAATTCAGTAGTGAAGGCCCCGTCAAATTC,
as shown in SEQ ID NO. 5;
and
ZWF1-R:
GCTCTAGACTAATTATCCTTCGTATCTTGTGGC,
as shown in SEQ ID NO. 6;

where the underlined parts represent added restriction enzyme digestion sites.

The examples involved the following medium formulas:

LB solid medium: 5 g of yeast powder, 10 g of peptone, and 10 g of sodium chloride were dissolved in deionized water, a resulting solution was diluted to 1 L. and a pH was adjusted to 7.0; and then 18 g of agar powder was added, and a resulting mixture was sterilized at 121° C. for 20 min.

LLB solid medium: 5 g of yeast powder, 10 g of peptone, and 5 g of sodium chloride were dissolved in deionized water, a resulting solution was diluted to 1 L, and a pH was adjusted to 7.0; and then 18 g of agar powder was added, and a resulting mixture was sterilized at 121° C. for 20 min.

YPD solid medium: 20 g of peptone, 10 g of yeast powder, 20 g of glucose, and 18 g of agar powder were mixed, and a resulting mixture was diluted to 1 L and sterilized at 115° C. for 20 min.

YPG medium: 20 g of peptone, 10 g of yeast powder, and 20 g of glycerin were mixed, and a resulting mixture was diluted to 1 L and sterilized at 115° C. for 20 min.

MD solid medium: 20 g of glucose and 18 g of agar powder were dissolved in 900 mL of water, and a resulting mixture was autoclaved at 115° C. for 20 min and then cooled to 60° C.; and then 13.4% of YNB (100 mL) and 0.02% of biotin (2 mL) were added.

YPDS solid medium: 20 g of peptone, 10 g of yeast powder, 20 g of glucose, 18 g of sorbitol, and 18 g of agar powder were mixed, and a resulting mixture was diluted to 1 L and sterilized at 115° C. for 20 min.

Fermentation medium: 85% phosphoric acid: 26.7 mL/L, CaSO₄: 0.93 g/L, K₂SO₄: 18.2 g/L, MgSO₄: 7.27 g/L, KOH: 4.13 g/L, Tween 80: 0.6 mL/L, glycerin: 40 g/L, and trace element: 10.8 mL, where the trace element was obtained by mixing 6.0 g of CuSO₄.5H₂O, 0.08 g of NaI, 3.0 g of MnSO₄.H₂O, 0.2 g of NaMoO₄.2H₂O, 0.02 g of H₃BO₃, 0.5 g of CoCl₂, 20.0 g of ZnCl₂, 65.0 g of FeSO₄.7H₂O, 0.2 g of biotin, and 5.0 mL of sulfuric acid and diluting a resulting mixture to 1 L.

The D-ethylgonendione used in the examples was purchased from Beijing Zizhu Pharmaceutical Co., Ltd; organic reagents such as ethyl acetate and chloroform were purchased from Tianjin Sixth Chemical Reagent Factory; TLC silica gel plates were purchased from Yantai Dexin Biotechnology Co., Ltd; molecular cloning reagents were purchased from Takara Biomedical Technology (Beijing) Co., Ltd; and other reagents not specified with sources were all purchased from Sangon Biotech (Shanghai) Co., Ltd.

### Example 1

1. Construction of the Recombinant Plasmid pPIC3.5K-PRH

RNA was extracted from P. raistrickii, and a cDNA library was obtained by reverse transcription. With PRH-F/R as primers, PCR amplification was conducted to obtain a PRH target gene fragment with EcoR I/Not I restriction enzyme digestion sites. The target fragment was digested with restriction endonucleases EcoR I/Not I, and the plasmid pPIC3.5K (Invitrogen) was digested with the same restriction endonucleases. The obtained PRH target gene fragment and the plasmid pPIC3.5K backbone were subjected to ligation at 22° C. for 2 h and then transformed into competent E. coli JM109. The E. coli was spread on an LB plate, and the recombinant plasmid pPIC3.5K-PRH was successfully obtained by screening for Amp resistance.

2. Construction of the Recombinant Plasmid pPICZ-ZWF1

Genomic DNA was extracted from S. cerevisiae INVSc1 (Invitrogen). With ZWF1-F/R as primers, PCR amplification was conducted to obtain a ZWF1 target gene fragment with EcoR I/Xba I restriction enzyme digestion sites. The target fragment was digested with restriction endonucleases EcoR I/Xba I, and the plasmid pPICZ (Invitrogen) was digested with the same restriction endonucleases. The obtained ZWF1 target gene fragment and the plasmid pPICZ backbone were subjected to ligation at 22° C. for 2 h and then transformed into competent E. coli JM109. The E. coli was spread on an LLB plate, and the recombinant plasmid pPICZ-ZWF1 was successfully obtained by screening for zeocin resistance.

3. Construction of the Recombinant Pichia pastoris (Engineered Strain Expressing Steroid 15α-Hydroxylase)

The recombinant plasmid pPIC3.5K-PRH in step 1 was linearized by restriction endonuclease Sal I and then electrotransformed into competent Pichia pastoris GS115 (Invitrogen), and the Pichia pastoris was cultivated according to a conventional method. G418 geneticin was used to screen a high-copy PRH strain. The recombinant plasmid pPICZ-ZWF1 in step 2 was linearized by restriction endonuclease Sac I and then electrotransformed into the competent PRH high-copy strain, and then the strain was cultivated according to a conventional method. The recombinant Pichia pastoris (engineered strain expressing steroid 15α-hydroxylase) was finally obtained by screening for zeocin resistance. FIG. 3 shows the construction of the recombinant Pichia pastoris, and FIG. 4 shows a PCR identification result of the genome of the obtained recombinant Pichia pastoris (engineered strain expressing steroid 15α-hydroxylase).

4. Establishment of a Process to Convert D-Ethylgonendione with a 5 L Fermentation System of the Recombinant Pichia pastoris (Engineered Strain Expressing Steroid 15α-Hydroxylase) at a High Substrate Loading

1) Cultivation of a seed suspension: the recombinant Pichia pastoris (15α-hydroxylase engineering strain) was inoculated on a YPD plate with 2 mg/mL G418 and 800 μg/mL bleomcyin, and then cultivated at 30° C. for 36 h; single colonies were picked and inoculated into a 250 mL Erlenmeyer flask with 50 mL of YPG liquid medium, and cultivated in a shaker for 24 h at 30° C. and 220 r/min to obtain a bacterial suspension, namely, a first-class seed culture; and 5 mL of the bacterial suspension was inoculated into a 250 mL Erlenmeyer flask with 100 mL of YPG liquid medium, and cultivated in a shaker for 18 h at 30° C. and 220 r/min until OD₆₀₀ was determined to be 12.

2) Preparation of a fermentation tank: a fermentation medium was prepared and added according to an initial filling volume of 2.5 L, thoroughly stirred, and sterilized at 121° C. for 30 min, and after the medium was cooled to 30° C., a pH was adjusted to 5.0 with ammonia water.

3) Inoculation: 200 mL of the seed suspension was inoculated into the tank, and fermentation was conducted at a temperature of 30° C., a pH maintained at 5.0, and a DO maintained at above 20%.

4) Bacterial growth stage: Fermentation was conducted at a temperature of 30° C. and a DO maintained at above 20%; and after glycerin in the fermentation medium was exhausted, the DO rose rapidly, and then a feed-batch cultivation stage started;

5) Feed-batch cultivation stage: 50% glycerin (12 ml of a trace element stock solution was added to each liter of glycerin) was fed at an initial rate of 19.92 mL/(L·h), and the feeding was stopped when the DO was lower than 20%; and after the glycerin was exhausted once again and the DO rose rapidly, the bacteria were starved for 1 h and then an induced enzyme production stage started.

6) Induction stage of enzyme production and D-ethylgonendione conversion: methanol (12 ml of a trace element stock solution was added to each liter of methanol in advance) was fed. The methanol was fed at an initial rate of 3.61 mL/(L·h), and the feeding was stopped when the DO was lower than 20%; after the DO rose rapidly, the feeding was restarted; after the feeding was conducted for 2 h, the feeding rate was increased to 7.22 mL/(L·h), and induction was further conducted for 2 h; then the feeding rate of methanol was increased to 10.89 mL/(L·h), and D-ethylgonendione (dissolved in methanol) was fed at the same time until D-ethylgonendione had a final concentration of 8 g/L; and conversion was conducted for 110 h to 130 h until the D-ethylgonendione was completely converted.

5. TLC Assay of a Product

Sample treatment: 1 mL of the fermentation broth obtained after the conversion in step 4 was completed was taken and added to a 1.5 mL centrifuge tube, 200 μL of ethyl acetate was added for extraction, and a resulting mixture was vigorously shaken for thorough mixing and then centrifuged at 12,000 r/min for 5 min to be separated into layers. About 2 cm to 4 cm of the upper ethyl acetate layer was pipetted using a capillary pipette with an inner diameter of 0.3 mm (10 cm in length) and spotted on a silica-gel chromatography plate. The sample was spotted at 1 cm from the edge of the chromatography plate, with a spacing of 0.5 cm among sample spots. A developing tank was saturated with a developing agent for 30 min before developing.

Chromatography: the silica gel plate spotted with the sample was put into the developing tank for developing, then the developing tank was covered and sealed, and when the frontier of the developing agent was close to the top, the plate spotted with the sample was taken out and dried. An ultraviolet (UV) detector was used to observe the color and size of chromatographic spots. The developing agent was a mixture of petroleum ether and ethyl acetate in a ratio of 5.5:4.5.

A TLC assay result of the product was shown in FIG. 4. The result showed that the D-ethylgonendione was almost converted completely by this strain.

6. HPLC Assay of the Conversion Product

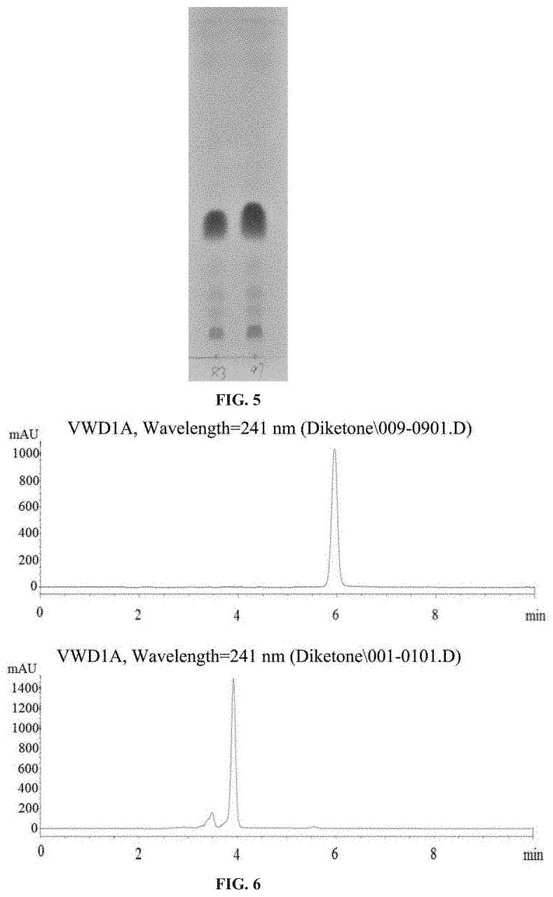

mL of the fermentation broth obtained after the conversion in step 4 was completed was added to a 50 mL centrifuge tube, then 10 mL of ethyl acetate was added, and a resulting mixture was fully shaken for extraction and centrifuged at 12,000 r/min for 2 min to obtain an ethyl acetate organic phase: the remaining bacterial solution was subjected to extraction twice; and organic phases were combined and subjected to HPLC detection. An HPLC chromatogram was shown in FIG. 5. The HPLC result further indicated the high conversion rate of the strain.

Chromatographic conditions: chromatographic column: C₁₈ column (4.6 mm DL×250 L mm, 5 μm): mobile phase: acetonitrile and water in a ratio of 80:20; flow rate: 0.8 mL/min; column temperature: 25° C.; injection volume: 10 μL; UV detector; and detection wavelength: 241 nm. The acetonitrile used in this method was chromatographically pure, and the water was deionized water that was filtered and ultrasonically treated.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. Recombinant Pichia pastoris, wherein the recombinant Pichia pastoris is obtained by inserting a Penicillium raistrickii (P. raistrickii) steroid 15α-hydroxylase gene PRH and a Saccharomyces cerevisiae (S. cerevisiae) glucose-6-phosphate dehydrogenase (G6PD) gene ZWF1 into a genome of Pichia pastoris;
the P. raistrickii steroid 15α-hydroxylase gene PRH is inserted into a His4 site of the genome of Pichia pastoris;
the S. cerevisiae G6PD gene ZWF1 is inserted into a 5′AOX1 site of the genome of Pichia pastoris;
the P. raistrickii steroid 15α-hydroxylase gene PRH has a nucleotide sequence shown in SEQ ID NO. 1; and
the S. cerevisiae G6PD gene ZWF1 has a nucleotide sequence shown in SEQ ID NO. 2.

2. A construction method of the recombinant Pichia pastoris according to claim 1, comprising the following steps:
S1. inserting the P. raistrickii steroid 15α-hydroxylase gene PRH into pPIC3.5K to obtain a first recombinant plasmid;
S2. inserting the S. cerevisiae G6PD gene ZWF1 into pPICZ to obtain a second recombinant plasmid; and
S3. linearizing the first recombinant plasmid with a restriction endonuclease Sal I and the second recombinant plasmid with a restriction endonuclease Sac I, and electrotransforming the plasmids into Pichia pastoris to obtain the recombinant Pichia pastoris;
wherein steps S1 and S2 can be conducted in any order.

3. The construction method according to claim 2, wherein the P. raistrickii steroid 15α-hydroxylase gene PRH is inserted into the pPIC3.5K at sites of EcoR I and Not I; and the S. cerevisiae G6PD gene ZWF1 is inserted into the pPICZ at sites of EcoR I and Xba I.

4. The construction method according to claim 2, wherein the Pichia pastoris in step S3 comprises Pichia pastoris GS115.

5. Use of the recombinant Pichia pastoris according to claim 1 in the efficient preparation of 15α-D-ethylgonendione, comprising the following steps:
1) inoculating the recombinant Pichia pastoris into a seed culture medium, and cultivating at 200 r/min and 28° C. to 30° C. until a resulting bacterial solution has OD₆₀₀ of 11 to 13 to obtain a starter culture;
2) inoculating the starter culture into a fermentation medium, and conducting fermentation cultivation at a temperature of 28° C. to 30° C. and a dissolved oxygen content of more than 20% until glycerin in the fermentation system is exhausted;
3) after the glycerin in the fermentation system is exhausted, feeding a glycerin-trace element aqueous solution into the fermentation system at a rate of 19.92 mL/(L·h), and stopping the feeding when a dissolved oxygen content is lower than 20%; further conducting fermentation cultivation until glycerin is exhausted once again; and with the glycerin being exhausted, starving bacteria for 1 h, wherein the glycerin-trace element aqueous solution comprises glycerin with a volume percentage of 45% to 55% and a 12 mL/L trace element aqueous solution;
4) after the bacteria are starved for 1 h, feeding a trace element-methanol solution into the fermentation system with a dissolved oxygen content of 100% for the first time at a rate of 3.61 mL/(L·h), and stopping the feeding when a dissolved oxygen content is lower than 20%; conducting a first induction cultivation for 2 h at 28° C. to 30° C.; and feeding the trace element-methanol solution into the fermentation system for the second time at a rate of 7.22 mL/(L·h) for 2 h; and
5) after the trace element-methanol solution is fed for the second time for 2 h, feeding a D-ethylgonendione-methanol solution at a rate of 10.89 mL/(L·h) until the D-ethylgonendione has a concentration of 8 g/L in the fermentation system; and with a dissolved oxygen content being controlled always at above 20%, conducting reaction at 28° C. to 30° C. until the D-ethylgonendione is completely converted;
wherein the fermentation medium in step 2) comprises the following components, with water as a solvent: 85% phosphoric acid: 26.7 mL/L, CaSO₄: 0.93 g/L, K₂SO₄: 18.2 g/L, MgSO₄: 7.27 g/L, KOH: 4.13 g/L, Tween 80: 0.6 mL/L, glycerin: 40 g/L, and a trace element aqueous solution with a volume percentage of 0.43%; and the fermentation medium has a pH of 5.0;
the trace element-methanol solution in step 4) is based on methanol and further comprises a 12 mL/L trace element aqueous solution;
the D-ethylgonendione-methanol solution in step 5) is based on methanol and further comprises a 12 mL/L trace element aqueous solution and 4 g/L to 5 g/L D-ethylgonendione; and
the trace element aqueous solution comprises the following components, with water as a solvent: CuSO₄.5H₂O: 6.0 g/L, NaI: 0.08 g/L, MnSO₄.H₂O: 3.0 g/L, NaMoO₄.2H₂O: 0.2 g/L, H₃BO₃: 0.02 g/L, CoCl₂: 0.5 g/L, ZnCl₂: 20.0 g/L, FeSO₄.H₂O: 65.0 g/L, biotin: 0.2 g/L, and sulfuric acid: 5.0 mL/L.

6. The use according to claim 5, wherein the seed culture medium in step 1) is YPG liquid medium.

7. The use according to claim 5, wherein an initial inoculation amount of the starter culture in step 2) is based on OD₆₀₀ after inoculation; and the OD₆₀₀ is 0.8 to 1.0.
